(19) United States
(12) Patent Application Publication
(10) Pub. No.: US 2016/0024545 A1
(43) Pub. Date: Jan. 28, 2016

(54) METHODS OF CONVERTING LIGNING AND USES THEREOF

(71) Applicant: SUGANIT SYSTEMS, INC., Reston, VA (US)

(72) Inventors: Anantharam Prasad DADI, Toledo, OH (US); Praveen PARIPATI, Reston, VA (US)

(21) Appl. No.: 14/776,161

(22) PCT Filed: Mar. 14, 2014
(86) PCT No.: PCT/US14/27374

§ 371 Date: Sep. 14, 2015

Related U.S. Application Data
(60) Provisional application No. 61/799,012, filed on Mar. 15, 2013.

(51) Int. Cl. C12P 19/14; C12P 19/02; C07C 27/04; C07H 3/02; C07C 1/22

(52) U.S. Cl. CPC C12P 19/14; C07H 3/02; C07C 1/22; C07C 27/04; C12P 19/02

(57) ABSTRACT

Method for creating valuable products from lignocellulosic biomass comprising sequential pretreatment of lignocellulosic biomass with ionic liquid followed by hydrothermal processing of the lignin.

### FIELD OF THE INVENTION

The present invention relates to a method converting lignin to chemicals.

### BACKGROUND OF THE INVENTION

Lignocellulose is the major structural component of plants and comprises cellulose, hemicellulose, and lignin. In lignocellulosic biomass, crystalline cellulose fibrils are embedded in a less well-organized hemicellulose matrix which, in turn, is surrounded by an outer lignin seal. Lignocellulosic biomass is an attractive feed-stock because it is an abundant, domestic, renewable source that can be converted to liquid transportation fuels, chemicals and polymers. The major constituents of lignocellulose are: (1) hemicellulose (20-30%), an amorphous polymer of five and six carbon sugars; (2) lignin (5-30%), a highly cross-linked polymer of phenolic compounds; and (3) cellulose (30-40%), a highly crystalline polymer of cellobiose (a glucose dimer).

Lignin is a complex, hydrophobic, cross-linked aromatic polymer. In nature, lignin is mainly found as an integral part of the cell wall, embedded in a carbohydrate polymer matrix of cellulose and hemicellulose. Isolation of native lignin is complicated, when at all possible. Lignins are polymers of phenylpropylene units, the exact composition of lignin varies widely with species. It has been found that not all lignin is homogenous in structure; it seems to consist of amorphous regions and structured forms. Lignin in higher cell walls is not amorphous. Novikova, et al. (2002) Appl. Biochem. Microbial. 38: 181-185. Both the chemical and three-dimensional structure of lignin is strongly influenced by the polysaccharide matrix. Houtman & Atalla (1995) Plant Physiol. 107: 997-984. Despite the fact that lignin is hydrophobic in character, molecular dynamic simulations have suggested that the hydroxyl and methoxyl groups in lignin precursors and oligomers may interact with cellulose microfibrils. The chemical structure of native lignin is essentially changed under high temperature and acidic conditions. At temperatures higher than 200° C., lignin has shown to be agglomerated into smaller particles and separated from cellulose. Tanahashi, et al. (1983) J. Biochem. 125: 728-736.

Extracting lignin from lignocellulosic biomass generally results in lignin fragmentation into numerous mixtures of irregular components. The generated lignin fractions, referred to as lignin, are difficult to elucidate and characterize. Thus, lignin are usually burned to produce heat and/or electricity within paper mills and biorefineries. Lignin lack consistency in their chemical and functional properties, they have complex molecular structures, and it is difficult to perform reliable routine analysis of the structural conformity and integrity of recovered lignin. EP 2435458 A1. As such, lignin have not been adopted for widespread use, rather the cost for producing and/or purifying lignin is uneconomical and therefore lignin are usually deposited as waste.

Methods of extracting lignin from lignocellulosic biomass are known in the art. Lignin may be recovered during or after pulping of lignocellulosic feedstocks. See EP 2435458 A1. Lignin may be extracted by the method of kraft pulping. See EP1002154. Lignin may be isolated by a method of alkaline pulping. See EP0091457. Lignin may be extracted using the process of acid hydrolysis. See EP0824616; EP1945823. Lignin may be extracted by a method of sulfite pulping. See EP0205778. Lignin may be extracted by a method of dissolving cellulose. See EP1654307. Lignin may be extracted by contacting the lignin (having a 1,1-diphenylpropan unit) with a metal oxide in a liquid medium and separating the metal oxide carrying the lignin. See EP 1900745 A1. Lignin may be extracted by pulping the feedstock with a selected organic solvent and acid catalyst (pH) for a selected period of time, separating the cellulosic solids fraction from the extractives liquid fraction; and recovering the lignin from an extractives liquid fraction. See U.S. Pat. No. 8,378,020 B1. Lignin may be extracted using methods of bonding a phenol derivative to the lignocellulose resource and, thereafter, contacting the lignocellulose resource with sulfuric acid, whereby lignin is separated from cellulose, because lignin has a bound phenol derivative. See EP 1022283 A1.

Efforts in converting lignin to its monomeric products have focused on technical lignins extracted from lignocellulosic biomass. Efforts have been advanced to depolymerize lignin through breakdown of alkyl-aryl ether linkages. However, this method has proven ineffective. There have also been methods advancing electro-catalytic oxidative cleavage of lignin. However, the products obtained were very low in yield and not economical. Oxidative depolymerization has also been advanced. Though this process yielded a high recovery, the process was uneconomical because it utilized high pressure, and very long reaction times. Lignin has also been depolymerized using 1-ethyl-3-methylimidazolium acetate. Varanasi, et al. (2013) Biotechnology for Biofuels. 6:14. However, it is limited to a low percentage of biomass load, where increasing the amount of biomass does not yield a higher percentage of monomeric products.

Therefore, there exists a need in the art for a more efficient method of extracting lignin from biomass and converting it to its constituent monomers and chemicals.

### SUMMARY OF THE INVENTION

In one embodiment, a method for treating a lignocellulosic biomass may comprise incubating a lignocellulosic biomass comprising lignin, cellulose, and hemicellulose in an ionic liquid (IL) for a sufficient time and temperature to swell the cellulose and hemicellulose by without dissolution of the biomass in the IL; washing the IL-incubated biomass comprising lignin, cellulose and hemicellulose with a liquid non-solvent for cellulose that is miscible with water and the IL; and contacting said swelled washed biomass comprising lignin, cellulose and hemicellulose with an aqueous buffer comprising enzymes capable of hydrolyzing both cellulose and hemicellulose to produce polysaccharides; recovering the lignin; and converting said lignin to chemicals.

In one embodiment, a method for extracting a monomeric compound from a lignin may comprise (a) mixing a biomass with an ionic liquid (IL) to swell said biomass and not dissolve said biomass in IL; (b) washing said treated biomass; (c) hydrolysis of said treated biomass; (d) separating the cellulosic and lignin fractions; (e) subjecting the lignin fraction to hydrothermal processing. In another embodiment, the method may further comprise electromagnetic (EM) heating of said swelled biomass after step (a).

In one embodiment, a method for conversion of the lignin of lignocellulosic biomass to chemicals may comprise (a) mixing biomass in an ionic liquid (IL) to swell said biomass and not dissolve said biomass in IL; (b) applying radio frequency (RF) heating to the swelled biomass to heat to a target temperature range; (c) applying ultrasonics, electromagnetic (EM), convective, conductive heating, or combinations thereof, to the swelled biomass to maintain the biomass at said target temperature range; (d) washing the treated biomass; (e) separating the cellulosic and lignin fractions; and (f) subjecting the lignin fraction to hydrothermal processing. In another embodiment, said target temperature range may be about 50-220° C.

In one embodiment, a method for disruption of the structure of a lignocellulosic biomass may comprise incubating a biomass in an ionic liquid (IL) and applying radiofrequency (RF) heating and ultrasonics, electromagnetic (EM), convective, conductive heating, or combinations thereof; washing the treated biomass; recovering the lignin; and subjecting the lignin fraction to hydrothermal processing.

In one embodiment, a method for conversion of the lignin of lignocellulosic biomass to chemicals may comprise hydrothermal processing of lignin.

In another embodiment, said lignocellulosic biomass may be agricultural residue, wood and forest residue, kudzu, red algae, herbaceous energy crop, plant biomass, or mixtures thereof. In another embodiment, the agricultural residue may be corn stover, wheat straw, bagasse, rice hulls, or rice straw. In yet another embodiment, the wood and forest residue may be pine, poplar, Douglas fir, oak, saw dust, paper/pulp waste, or wood fiber. In another embodiment, the herbaceous energy crop may be switchgrass, reed canary grass, or miscanthus.

In one embodiment, the hydrothermal processing may comprise increased pressure. In another embodiment, said increase pressure may be at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 50-100, or 50-150 ATM. In yet another embodiment, the pressure may be about 10-100, 20-80, 10-120, or 70-120 ATM.

In one embodiment, the hydrothermal processing may comprise increased temperature. In another embodiment, said temperature may be about 100, 110, 120, 130, 140, 150, 160, 170, 180, 190, 200, 210, 220, 230, 240, 250, 260, 270, 280, 290, 300, 200-250, 200-300, or 250-300° C. In another embodiment, the temperature may be about 100-300 ° C., 100-350 ° C., 200-300 ° C., 250-350° C., or 300-350 ° C.

In one embodiment, hydrothermal processing may be for about 1, 2, 3, 4, 5, 6, 7, 8, 9, 10, 11, 12, 13, 14, 15, 16, 17, 18, 19, 20, 10-20, 10-15, or 1-10 minutes. In another embodiment, hydrothermal processing may be for about 0-3, 1-3, 3-10, 1-10, or 10-15 minutes.

In another embodiment, the hydrothermal processing may comprise conversion in presence of aromatic or aliphatic alcohols/acids under mild acidic or basic medium.

In another embodiment, the hydrothermal processing may be catalytically conducted in a single or sequential steps to produce oxygenated products, deoxygenated products and/or dehydrogenated products.

In one embodiment, the method may further comprise treating said lignin to convert the lignin to its constituent monomers and chemicals. In another embodiment, the method may further comprise chemical analysis of said constituent monomers and chemicals. In another embodiment, the chemical analysis may be gas chromatography—mass spectrophotometry, gas chromatography—infrared spectroscopy, liquid chromatography—mass spectrometry, liquid chromatography—NMR spectroscopy, or liquid chromatography—infrared spectroscopy.

In one embodiment monomer may be toluene; phenol; phenol, 2-methyl; phenol, 3-methyl; indan, 1-methyl; phenol, 2-methoxy; phenol, 4-methoxy-3-methyl; naphalene; 2-methoxy-5-methylphenol; phenol, 2-methoxy-4-methyl; 3,4-dimethoxytoluene; phenol, 3,4-dimethoxy; 1,2-Benzenediol, 3-methoxy; Phenol, 4-ethyl-2-methoxy; Naphthalene, 2-methyl; Naphthalene, 1-methyl; 2-methoxy-4-vinylphenol; Benzene, 4-ethyl-1,2-dimethoxy; 1,2,4-trimethoxybenzene; Phenol, 2,6-dimethoxy; 3-allyl-6-methoxyphenol; Phenol, 2-methoxy-4-propyl; Naphthalene, 1-ethyl (or 2-ethyl); Vanillin; Benzene, 1,2,3-trimethoxy,5-methyl; Pheno1,2-methoxy-4-(1-propenyl); Biphenylene; 3-Hydroxy-4-methoxybenzoic acid; Acenaphthene; Ethanone, 1-(2,6-dihydroxy-4-methoxyphenyl); 1-Isopropenylnaphthalene; Hexadecane; Phenol, 2,6-dmethoxy-4-(2-propenyl); Phenol, 2,6-dimethoxy-4-(2-propenyl); Benzaldehyde, 4-hydroxy-3,5-dimethoxy; 8-Heptadecene; Benzeoic acid, 3,4,5-trimethoxy-, methyl ester; Ethanone, 1-(4-hydroxy-3,5-dimethoxyphenyl); Anthracene; Phenanthrene, 1-methyl; Anthracene, 1-methyl; Phenanthrene, 1-methyl; Anthracene, 9-ethyl; Phenanthrene, 4,5-dimethyl; Fluoranthene; Pyrene; Acephenanthrylene, 4,5-dihydro; Benzo[k]fluoranthene; Stigmastan-3,4-dien; 9,10-anthracenedione, 1,8-dichloro; Benzo[ghi]perylene; Coronene; 1-hydroxy-2-butanone; 2-Furanmethanol; Butyrolactone; 1H-Imidazole, 1-methyl; Phenol, 2-methoxy; 1,2-Benzenediol, 3-methoxy; 2-methoxy-4-vinylphenol; Phenol, 2,6-dimethoxy; Phenol, 3,4-dimethoxy; 3-hydroxy-4-methoxybenzoic acid; Benzaldehyde, 4-hydroxy-3,5-dimethoxy; Phenol, 2,6-dimethoxy-4-(2-propenyl); Ethanone, 1-(4-hydroxy-3,5dimethoxyphenyl); 2-Pentanone, 1-(2,4,6-trihydroxyphenyl); Butyrolactone; 1H-Imidazole, 1-methyl; Phenol, 2-methoxy; 1,2-Benzenediol, 3-methoxy; Phenol, 4-ethyl-2-methoxy; 2-methoxy-4-vinylphenol; Pheno1,2,6-dimethoxy; Phenol, 3,4-dimethoxy; 3-hydroxy-4-methyoxy-benzoic acid; 4-methyl-2,5-dimethoxybenzaldehyde; Phenol, 2,6-dimethyoxy-4-(2-propenyl); Benzaldehyde, 4-hydroxy-3,5-dimethoxy; Phenol, 2,6-dimethyoxy-4-(2-propenyl); Ethanone,1-(4-hydroxy-3,5-dimethoxyphenyl); 2-pentanone, 1-(2,4,6-trihydroxyphenyl); or combinations thereof.

In another embodiment, the monomer may be 1-propanol, 2 methoxy; Butyrolactone; Pentanoic Acid 4 oxo methyl ester; Hexanal 2-ethyl; Phenol, 2 methoxy; Phenol 2 methoxy-4 methyl; 1,4-Benzenediol, 2-methoxy; Phenol 4-ethyl 2 methoxy; Phenol, 2,6-dimethoxy; Phenol, 2-etmoxy-4 propyl; 1,3-benzenediol 4 ethyl; Benzoic Acid, 4-hydroxy-3methoxy; 1,3-Benzenediol, 4 propyl; Ethanone,1-(4-hydroxy-3-methoxy phenyl); Benzene, 1,2,3-Trimethoxy-5 methyl; 2 Propanone,1-(4-hydroxy-3-methoxy phenol; Homovanillyl Alcohol; 3,4 Dimethoxyphenyl acetone; Benzeneacetic acid, 4-hydroxy 3 methoxy; Vanillacetic acid; Ethyl homovanillate; Ethanone 1-(4-hydroxy-3,5-dimethoxy phynyl); Phenol, 2-methoxy-4-propyl; or combinations thereof.

In another embodiment, the chemical may be phenol, guaiacol, syringol, eugenol, catechol, vanillin, vanillic acid, syringaldehyde, benzene, toluene, xylene, styrene, biphenyl, cyclohexane, or combinations thereof.

In on embodiment, the biomass may be subjected to additional heating with agitation, ultrasonics heating, electromagnetic (EM) heating, convective heating, conductive heating, microwave irradiation, or a combination thereof. In another embodiment, the electromagnetic (EM) heating may be radiofrequency (RF) heating.

In another embodiment, the heating may comprise at least two phases, a first phase comprising application of electromagnetic (EM) heating, variable frequency heating, radiofrequency (RF) heating, or a combination thereof, and a second phase comprising application of ultrasonics, electromagnetic (EM), convective, conductive heating, or combinations thereof. In yet another embodiment, the first phase may comprise a variable frequency in the electromagnetic spectrum. In another embodiment, application of radiofrequency heating may be for about at least 5-10 seconds, 1-30 minutes, 5-30 minutes, or 20-240 minutes. In another embodiment, application of ultrasonics, electromagnetic (EM), convective, conductive heating, or combinations thereof, may be for about at least 3-30 minutes, 5-30 minutes, or 3-4 hours.

In one embodiment, electromagnetic energy may be applied at a power of 100-1000 W, 1 KW-10 KW, or 5 KW-1 MW.

In one embodiment, radiofrequency may comprise a frequency between about 1-900 MHz, 300 kHz-3 MHz, 3-30 MHz, 30-300 MHz, 13, 13.56, 27, 27.12, 40, or 40.68 MHz. In another embodiment, radiofrequency may penetrate the biomass to about 0.001 to 2.0 meters thickness. In another embodiment biomass may be treated with radiofrequency for at least about 1 minute to 100 hours, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

In one embodiment, biomass may be heated to a temperature of at least about 1-300° C., 50° C-100° C., 60° C-130° C., 80° C-175° C., or 100° C-240° C.

In one embodiment, the method may further comprise washing the treated biomass. In another embodiment, washing may comprise washing the biomass with a liquid non-solvent for cellulose that may be miscible with water and the ionic liquid (IL). In another embodiment, the liquid non-solvent used for washing may be water, an alcohol, acetonitrile or a solvent which dissolves the IL and thereby extracts the IL from the biomass. In another embodiment, the alcohol may be ethanol, methanol, butanol, propanol, or mixtures thereof. In another embodiment, the ionic liquid may be recovered from the liquid non-solvent by a method selected from one or more of activated charcoal treatment, distillation, membrane separation, electro-chemical separation techniques, sold-phase extraction liquid-liquid extraction, or a combination thereof. In another embodiment, the ionic liquid may be recovered from the liquid non-solvent by application of electromagnetic heating. In another embodiment, the ionic liquid may be recovered from the liquid non-solvent by application of radiofrequency heating, that dehydrates the ionic liquid.

In on embodiment, the method may further comprise reusing the recovered IL for treating more biomass. In another embodiment, at least 90, 91, 92, 93, 94, 95, 96, 97, 98, or 99% of the IL may be recovered.

In one embodiment, the ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%.

In one embodiment, the biomass may be subjected to additional heating with intermittent agitation during heating.

In one embodiment, the ionic liquid may be molten at a temperature ranging from about 10° C. to 160° C. and may comprise cations or anions. In another embodiment, the ionic liquid may comprise a cation structure that includes ammonium, sulfonium, phosphonium, lithium, imidazolium, pyridinium, picolinium, pyrrolidinium, thiazolium, triazolium, oxazolium, or combinations thereof. In another embodiment, the ionic liquid may comprise a cation selected from imidazolium, pyrrolidinium, pyridinium, phosphonium, ammonium, or a combination thereof.

In one embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the method may be a continuous process. In another embodiment, the method may be a batch process.

In one embodiment, the conditions of biomass undergoing radiofrequency (RF) heating may monitored by sensors. In another embodiment, conditions of biomass undergoing RF heating may monitored by a liquid flow rate sensor, thermocouple sensor, temperature sensor, salinity sensor, or combinations thereof.

In one embodiment, the method may comprise adjusting the amount of ionic liquid, the time of incubation, or the temperature of the biomass.

In one embodiment, the method may further comprise treating said treated lignocellulosic biomass with biochemical reagents. In another embodiment, said biochemical reagent may be an enzyme. In another embodiment, the enzyme may convert the cellulose and hemicellulose to sugar. In another embodiment, the sugar may be a hexose and pentose sugar.

### BRIEF DESCRIPTION OF THE DRAWINGS

FIG. 1 depicts an exemplary method for processing biomass comprising mixing with ionic liquid, heating by radio frequency irradiation to reach a target temperature range, optionally repeated, maintaining the temperature of the swelled biomass using of ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeated, washing the biomass, optionally recovering the IL and dehydrating the IL by application of radiofrequency heating, hydrolysis (e.g., addition of celluase and hemicellulases) of the cellulose and hemicellulose to their constituent monomeric sugars (e.g., five and six carbon sugars), optionally recovery of the added enzymes, separation of the hydrolystate stream comprising sugars for further processing to produce chemicals or biofuels and the residual solids comprising lignin for further processing to produce chemicals by hydrothermal processing. The enzymes may be reclaimed and reused.

FIG. 2A depicts an analysis of lignin separation by Gas Chromatography—Mass Spectrophotometry.

FIG. 2B depicts an analysis of lignin separated by Gas Chromatography—Mass Spectrophotometry including deoxygenated chemicals.

FIG. 3 depicts exemplary cation and anion components of ionic liquids.

### DETAILED DESCRIPTION OF THE PREFERRED EMBODIMENTS

In order that the invention herein described may be fully understood, the following detailed description is set forth. Various embodiments of the invention are described in detail and may be further illustrated by the provided examples. Additional viable variations of the embodiments can easily be envisioned.

### Definitions

Unless defined otherwise, all technical and scientific terms used herein have the same meaning as those commonly understood by one of ordinary skill in the art to which this invention belongs.

As used in the description herein and throughout the claims that follow, the meaning of “a,” “an,” and “the” includes plural reference unless the context clearly dictates otherwise.

“Biomass,” as used herein, refers broadly to any biological material. Biomass encompasses substrates containing organic components which can be used in production of renewable fuels, chemicals and materials such as ethanol, butanol, lactic acid, gasoline, biodiesel, methane, hydrogen, plastics, composites, protein, drugs, fertilizers or other components thereof. Biomass may be agricultural residues, optionally corn stover, wheat straw, bagasse, rice hulls, or rice straw; wood and forest residues, optionally pine, poplar, douglas fir, oak, saw dust, paper/pulp waste, or wood fiber; feedstock (e.g., woody biomass and agricultural biomass); kudzu; red algae; cellulose, lignin, herbaceous energy crops, optionally switchgrass, reed canary grass, or miscanthus; lingocellulosic biomass, optionally comprising lignin, cellulose, and hemicellulose; plant biomass; or mixtures thereof. Biomass may be lignocellulosic biomass comprising cellulose, hemicellulose, and lignin.

“Electromagnetic energy (EM),” as used herein, refers broadly to a form of energy that is reflected or emitted from objects in the form of electrical and magnetic waves that can travel through space. There are many forms of electromagnetic energy including gamma rays, x rays, ultraviolet radiation, visible light, infrared radiation, microwaves, and radio waves (radiofrequency).

“Ionic liquids” as used herein, refers broadly to room temperature liquids that contain only ions and are molten salts stable up to 300° C. Sheldon (2001) Chem.Commun. 23: 2399-2407.

“Lignocellulosic biomass” as used herein, refers broadly to plant biomass that is composed of cellulose, hemicellulose, and lignin. The carbohydrate polymers (e.g., cellulose and hemicelluloses) are tightly bound to the lignin. Lignocellulosic biomass can be grouped into four main categories: agricultural residues (e.g., corn stover and sugarcane bagasse), dedicated energy crops, wood residues (e.g., sawmill and paper mill discards), and municipal paper waste.

“Lignin,” as used herein, refers broadly to a highly cross-linked polymer of phenolic compounds deposited in the cell walls of many plants.

“Pretreatment of biomass,” as used herein, refers broadly to a process of changing the physiochemical structure of biomass to make it amenable for efficient conversion to their monomeric valuable products.

“Radiofrequency (RF) heating,” as used herein, refers broadly to application of electromagnetic field to biomass/products/dielectric materials at frequencies from about 1-300 MHz.

“Ultrasonics” or “ultrasonic waves,” as used herein, refers broadly to sound waves (mechanical waves) with high frequency about between 15 kHz to 40 kHz (e.g., about 20 kHz) and low amplitude about between 0.0001-0.025 mm. Sequential Ionic Liquid Pretreatment of Lignocellulosic Biomass followed by Conversion of Lignin to Chemicals

The present invention provides a method for the sequential treatment of lignocellulosic biomass to yield useful chemicals comprising the combination of ionic liquid pretreatment followed by hydrothermal processing of lignin.

The lignocellulosic processing strategy employs sequential ionic liquid pretreatment followed by hydrothermal processing of lignin which (a) can be used for treating any lignocellulosic biomass substrates, (b) results in efficient lignin fraction generation, (c) enables economic recovery of catalysts and chemicals. The inventors surprisingly discovered that the combination of ionic liquid pretreatment with hydrothermal processing of lignin which expectantly results in high yield of chemicals from lignin.

### Pretreatment of Biomass

Methods for the pretreatment of biomass are known in the art including ionic liquid pretreatment by dissolution, ionic liquid swelling, and alkaline treatment including Kraft processing.

Biomass pretreatment by ionic liquids where the biomass is swollen with ionic liquids, but not dissolved is described in U.S. Pat. No. 8,030,030. Biomass pretreatment by ionic liquids where the biomass is swollen with ionic liquids, but not dissolved, and heated with radiofrequency heating is described in U.S. Provisional Patent Application No. 61/663,315. The lignin obtained by these methods is closer to a native state of lignin and more amenable to conversion than lignin obtained by other methods.

An additional method of biomass pretreatment by ionic liquids where the biomass is swollen with ionic liquids and further treated according to U.S. Patent Application, referenced by Attorney-Docket number 73368.000029, is herein incorporated by reference.

Biomass pretreatment by ionic liquids where the biomass is dissolved in an ionic liquid is described in U.S. Patent Application Publication No. 2005/017252 and Varanasi, et al. (2013) Biotechnology for Biofuels 6:14. However, the lignin produced by these methods is of a lower molecular weight than methods where the biomass is swollen but not dissolved. This implies that the lignin is a fractured polymer and is less intact than methods where the biomass is swollen but not dissolved.

Biomass pretreatment by Kraft processing is well-known in the art, however, the lignin produced by Kraft processing results in lignosulfonates (sulfonated lignin). The lignin obtained by Kraft processing is less desirable because of the high level of sulfur.

### Ionic Liquid (IL)

Ionic liquids are liquids at room temperature and may contain only ions and are molten salts stable up to 300° C. See Sheldon (2001) Chem.Commun. 23: 2399-2407. They contain cations which are usually organic compounds and anions of inorganic or organic components such that the resulting salts are asymmetric. Because of poor packing associated with the asymmetric nature of ILs, crystal formation is inhibited and ILs remain liquids over a wide range of temperatures. A wide range of anions and cations can be employed to generate ILs with varied melting points, viscosities, thermal stabilities and polarities. Examples of some of the cations currently used include ammonium, sulfonium, phosphonium, lithium, imidazolium, pyridinium, picolinium, pyrrolidinium, thiazolium, triazolium oxazolium, or combinations thereof. Murugesan & Linhardt (2005) Current Organic Synthesis 2: 437-451. Ionic liquids are also liquid at <100° C., broad liquid range, almost no vapor pressure, high polarity, high dissolving power for organic and inorganic materials, good thermal, mechanical, and electrochemical stability, high heat capacity, non-flammable, and electrical conductivity.

Ionic liquids have extremely low volatility and when used as solvents, they do not contribute to emission of volatile components. In this sense they are environmentally benign solvents. ILs have been designed to dissolve cellulose and lignocellulose. Following dissolution, cellulose can be regenerated by the use of anti-solvents. However, the complete dissolution of lignocellulosic materials (particularly woods) in ILs is harder and, even partial dissolution, requires very long incubation of biomass in IL at elevated temperatures. Even then, a high yield of cellulose is not generally achieved after regeneration. Fort, et al. (2007) Green. Chem. 9: 63.

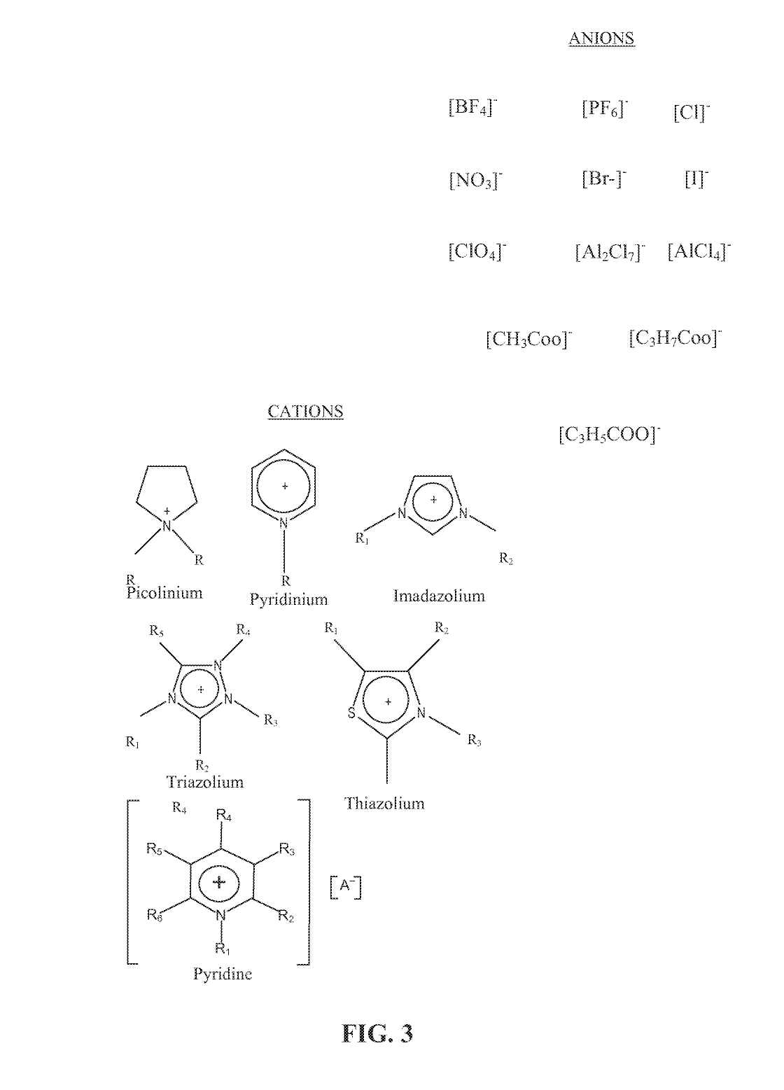

The present invention differs from the classic approach to the use of ionic liquids in that the aim is not to dissolve lignocellulose, but rather to contact it with the IL for times sufficient to mainly disrupt lignin sheathing and swell the remaining biomass structure significantly (at least 30%) but not dissolve the lignocellulose and further apply radio frequency heating. This combination treatment enables the subsequent enzymatic hydrolysis process to proceed in a relatively short period of time as well as give quantitative yields of glucose and high yields of pentose sugars. Any ionic liquid capable of disrupting the hydrogen bonding structure to reduce the crystallinity of cellulose in the biomass can be used in the treatment methods described herein, and may comprise a cation structure that includes imidazolium, pyrroldinium, pyridinium, phosphonium, ammonium, or a combination thereof and all functionalized analogs thereof. For example, the structure of triazolium as shown in FIG. 3 wherein each of R1, R2, R3, R4, and R5 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein an alkene group may be a halide, hydroxide, formate, acetate, propionate, butyrate, any functionalized mono- or di-carboxylic acid having up to a total of 10 carbon atoms, succinate, lactate, aspartate, oxalate, trichloroacetate, trifluoroacetate, dicyanamide, or carboxylate. Another example of the structure of IL is shown in FIG. 3 pyridine wherein each of R1, R2, R3, R4, R5, and R6 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A may be a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate. The halide can be a chloride, fluoride, bromide or iodide.

Also an ionic liquid mixture with a composition described by Equation 1 may be used in the methods and systems described herein.

$${\sum }_{n = 1}^{20} {{[ {C}^{+} ]}_{n} [ {A}^{-} ]}_{n}$$

C⁺ denotes the cation of the IL and A⁻ denotes the anionic component of the IL In Equation 1. Each additional IL added to the mixture may have either the same cation as a previous component or the same anion as a previous component, of differ from the first only in the unique combination of the cation and anion. For example, consider below the five component mixture of ILs in which common cations and anions are used, but each individual IL component is different:

[BMIM⁺][Cl⁻]+[BMIM⁺][PF₆₋]+[EMIM⁺][Cl⁻]+[EM−IM⁺][PF₆₋]+[EMIM⁺][BF₄⁻]

The final mixture of ionic liquids will vary in the absolute composition as can be defined by the mole percent of various functionalized cations and anions. Therefore, the mixture may be comprised of varying weight percentages of each utilized component, as defined by Equation 1. The use of several such representative solvents for treating biomass may be 1-Ethyl-3-Methylimidazolium Propionate (EMIM-Pr) as described in U.S. Pat. No. 8,030,030. Also the ionic liquid 1-(4-sulfonic acid) butyl-3-methylimidazolium hydrogen sulfate may be used.

The ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%. Also, the ionic liquid may be recovered and reused.

### Recovery of IL/Dehydration of IL

The wash effluent may be collected and the ionic liquid dehydrated by the application of RF energy. The RF energy heats IL faster than it heats water because of a stronger dipole moment in IL. Without being bound to a specific theory, the inventors surprisingly discovered that the ions try to align with the electromagnetic (EM) (e.g., radiofrequency) waves, always changing a dipole moment. The IL heated by RF acts as a substrate for the water to heat and evaporate from the IL wash effluent. Thus, the wash effluent comprising a solvent and ionic liquid may be heated using RF energy. The RF energy drive off the water which may be collected and removed from the wash. The resultant ionic liquid is thus dehydrated (e.g., the water has been removed) and may be reused. See U.S. Provisional Patent Application No. 61/663,315.

### Hydrothermal Conversion of Lignin

The lignin obtained by methods comprising ionic liquid pretreatment where the biomass is swelled with the ionic liquid but not dissolved may undergo hydrothermal processing to convert the lignin to its constituent monomers and chemicals.

The hydrothermal processing may be performed under increased pressure. The pressure may be greater than about 1 ATM, 10, 20, 30, 40, 50, 60, 70, 80, 90, 100, 60-150, 10-150 ATM. The pressure may be about 10-100 ATM. The pressure may be about 20-80 ATM. The pressure may be about 10-120 ATM. The pressure may be about 70-120 ATM.

The hydrothermal processing may be performed under increased temperature. The temperature may be about 100, 110, 120, 130, 140, 150, 160, 170, 180, 190, 200, 210, 220, 230, 240, 250, 260, 270, 280, 290, 300° C., 200-250° C., 200-300° C., 250-300° C., or 250-350 ° C. The temperature may be about 1000-300 ° C. The temperature may be about 100-350 ° C. The temperature may be about 2000-300 ° C. The temperature may be about 250-350 ° C. The temperature may be about 300-350 ° C.

The hydrothermal processing may be performed for about 1-60 minutes, 1-30 minutes, 1-15 minutes, 1 minute, 1-5 minutes, 10 minutes, or 15 minutes. The hydrothermal processing may be performed for about 1-10 minutes. The hydrothermal processing may be performed for about 1-3 minutes. The hydrothermal processing may be performed for about 0-3 minutes.

The hydrothermal processing may be performed in presence of aromatic or aliphatic alcohols/acids under mild basic or acidic conditions.

The hydrothermal processing may be performed in a batch or sequential mode in the presence of catalysts to produce (i) oxygenated products, (ii) deoxygenated products and or dehydrogenated products.

The separation process comprises hydrothermal processing. In one embodiment, hydrothermal processing depolymerizes lignin into its monomeric products. In an embodiment, hydrothermal processing is carried out at pressures less than or equal to 200 ATM. In an embodiment, hydrothermal processing is carried out at temperatures less than 300° C., preferably between 250° C., and 300° C. In an embodiment, lignin is subjected to hydrothermal processing for less than 10 minutes, preferably less than 1 minute.

The hydrothermal processing may comprise conversion in presence of aromatic or aliphatic alcohols/acids under mild acidic or basic medium.

The hydrothermal processing may be catalytically conducted in a single or sequential steps to produce oxygenated products, deoxygenated products and/or dehydrogenated products.

The separated lignin may be further analyzed using a chemical analytical method comprising: gas chromatography—mass spectrophotometry, gas chromatography—infrared spectroscopy, liquid chromatography—mass spectrometry, liquid chromatography—NMR spectroscopy, or liquid chromatography—infrared spectroscopy.

In one embodiment, the carrier gas for gas chromatography—mass spectrophotometry comprises helium, nitrogen, hydrogen, or argon.

Various chemicals that may be obtained from the hydrothermal process comprise, for example: toluene; phenol; phenol, 2-methyl; phenol, 3-methyl; indan, 1-methyl; phenol, 2-methoxy; phenol, 4-methoxy-3-methyl; naphalene; 2-methoxy-5-methylphenol; phenol, 2-methoxy-4-methyl; 3,4-dimethoxytoluene; phenol, 3,4-dimethoxy; 1,2-Benzenediol, 3-methoxy; Phenol, 4-ethyl-2-methoxy; Naphthalene, 2-methyl; Naphthalene, 1-methyl; 2-methoxy-4-vinylphenol; Benzene, 4-ethyl-1,2-dimethoxy; 1,2,4-trimethoxybenzene; Phenol, 2,6-dimethoxy; 3-allyl-6-methoxyphenol; Phenol, 2-methoxy-4-propyl; Naphthalene, 1-ethyl (or 2-ethyl); Vanillin; Benzene, 1,2,3-trimethoxy,5-methyl; Pheno1,2-methoxy-4-(1-propenyl); Biphenylene; 3-Hydroxy-4-methoxybenzoic acid; Acenaphthene; Ethanone, 1-(2,6-dihydroxy-4-methoxyphenyl); 1-Isopropenylnaphthalene; Hexadecane; Phenol, 2,6-dmethoxy-4-(2-propenyl); Phenol, 2,6-dimethoxy-4-(2-propenyl); Benzaldehyde, 4-hydroxy-3,5-dimethoxy; 8-Heptadecene; Benzeoic acid, 3,4,5-trimethoxy-, methyl ester; Ethanone, 1-(4-hydroxy-3,5-dimethoxyphenyl): Anthracene; Phenanthrene, 1-methyl; Anthracene, 1-methyl; Phenanthrene, 1-methyl; Anthracene, 9-ethyl; Phenanthrene, 4,5-dimethyl; Fluoranthene; Pyrene; Acephenanthrylene, 4,5-dihydro; Benzo[k]fluoranthene; Stigmastan-3,4-dien; 9,10-anthracenedione, 1,8-dichloro; Benzo[ghi]perylene; Coronene; 1-hydroxy-2-butanone; 2-Furanmethanol; Butyrolactone; 1H-Imidazole, 1-methyl; Phenol, 2-methoxy; 1,2-Benzenediol, 3-methoxy; 2-methoxy-4-vinylphenol; Phenol, 2,6-dimethoxy; Phenol, 3,4-dimethoxy; 3-hydroxy-4-methoxybenzoic acid; Benzaldehyde, 4-hydroxy-3,5-dimethoxy; Phenol, 2,6-dimethoxy-4-(2-propenyl); Ethanone, 1-(4-hydroxy-3,5dimethoxyphenyl); 2-Pentanone, 1-(2,4,6-trihydroxyphenyl); Butyrolactone; 1H-Imidazole, 1-methyl; Phenol, 2-methoxy; 1,2-Benzenediol, 3-methoxy; Phenol, 4-ethyl-2-methoxy; 2-methoxy-4-vinylphenol; Pheno1,2,6-dimethoxy; Phenol, 3,4-dimethoxy; 3-hydroxy-4-methyoxy-benzoic acid; 4-methyl-2,5-dimethoxybenzaldehyde; Phenol, 2,6-dimethyoxy-4-(2-propenyl); Benzaldehyde, 4-hydroxy-3,5-dimethoxy; Phenol, 2,6-dimethyoxy-4-(2-propenyl); Ethanone,1-(4-hydroxy-3,5-dimethoxyphenyl); or 2-pentanone, 1-(2,4,6-trihydroxyphenyl).

Various chemicals that may be obtained from the hydrothermal process comprise, for example: 1-propanol, 2 methoxy; Butyrolactone; Pentanoic Acid 4 oxo methyl ester; Hexanal 2-ethyl; Phenol, 2 methoxy; Phenol 2 methoxy-4 methyl; 1,4-Benzenediol, 2-methoxy; Phenol 4-ethyl 2 methoxy; Phenol, 2,6-dimethoxy; Phenol, 2-etmoxy-4 propyl; 1,3-benzenediol 4 ethyl; Benzoic Acid, 4-hydroxy-3methoxy; 1,3-Benzenediol, 4 propyl; Ethanone,1-(4-hydroxy-3-methoxy phenyl); Benzene, 1,2,3-Trimethoxy-5 methyl; 2 Propanone,1-(4-hydroxy-3-methoxy phenol; Homovanillyl Alcohol; 3,4 Dimethoxyphenyl acetone; Benzeneacetic acid, 4-hydroxy 3 methoxy; Vanillacetic acid; Ethyl homovanillate; Ethanone 1-(4-hydroxy-3,5-dimethoxy phynyl); or Phenol, 2-methoxy-4-propyl.

The separation process may also comprise column chromatography, high performance liquid chromatography, thin layer chromatography, size exclusion chromatography, or combinations thereof. In an embodiment, the separation process allows each compound fraction to elute at a specific retention time with a particular intensity, depending on the concentration of that compound in the lignin. These compounds may be commercially utilized.

The lignin extracted by the methods described herein may be used in cement and concrete, antioxidant, asphalt, animal feed pellets, animal feed molasses additives, road binder/dust control, pesticides, oil well drilling muds, adhesives, resins and binders, wallboard, dispersants, emulsifiers and wetting agents, agglomerants, chelants, leather treatment, anti-bacterial activity, lead acid batteries, oil recovery, water treatment, industrial cleaners, emulsion stabilizers, carbon black, inks and azo pigments, dyestuffs, micronutrients, fertilizers bricks, refractories and ceramic additives, ore processing, or kitty litter.

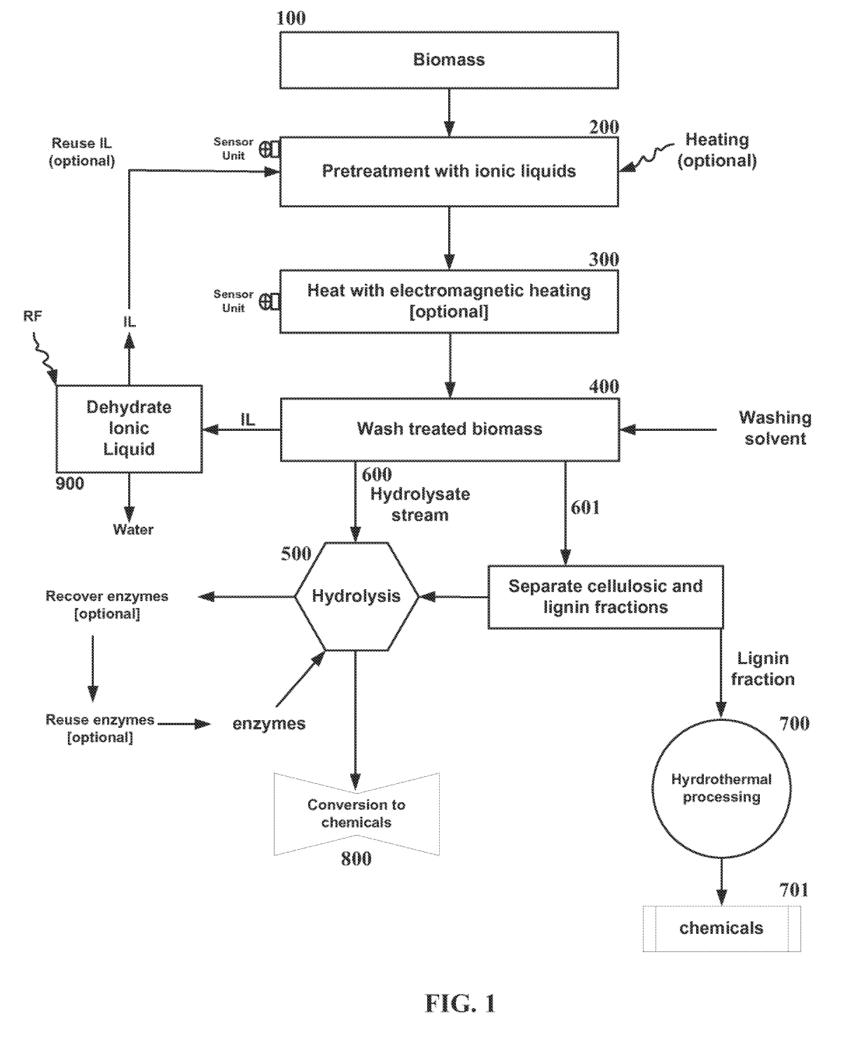

Proceeding now to a description of the drawings, FIG. 1 shows an exemplary series for carrying out steps of a method of the present invention.

One of the following representative ionic liquids 1-n-butyl-3-methylimidazolium chloride (BMIMCD/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with small particles of biomass 100 (e.g., dry corn stover or poplar (−20+80 mesh sized particles)] for varying times (about 5 minutes to 8 hours) 200. Incubation with biomass may be carried out using electromagnetic (EM) (e.g., radiofrequency) heating and ultrasonics, electromagnetic (EM) (e.g., radiofrequency), convective, conductive heating, or combinations thereof at about 50° C. to 200° C. as long as the ionic liquid is in molten state during incubation 300. The conditions may be monitored by use of sensors and adjusted to maintain conditions. The biomass may be heated with RF heating at about 27 mHz for at least about 5 seconds to 2 hours. The swelled biomass/IL may then be heated using ultrasonics, electromagnetic (EM) (e.g., radiofrequency), convective, conductive heating, or combinations thereof for about at least 3-30 minutes or 3-4 hours. The conditions may be monitored and adjusted to maintain uniform heating and sufficient penetration of the biomass by the RF waves. Steps 200, 300, and/or 400 may be repeated. Further, steps 300 and/or 400 may be carried out in batch or continuous form. The goal of treatment 300 is not achieving any dissolution of lignocellulose, but heating the swelled biomass for sufficient time to redistribute lignin and swell the remaining biomass structure to enhance the hydrolysis rate and conversion of cellulose and hemicellulose to their constituent sugars and release lignin 500.

The treated biomass may then be contacted with one of the representative wash-solvents, namely, methanol/ethanol/water/acetonitrile/butanol/propanol 400. The wash-solvent mixes with the IL (in all proportions) and hence is able to extract it from the incubated biomass. The treated biomass may then be separated from the ionic liquid/wash solvent solution by centrifugation. The hydrolysate stream of the biomass, stripped off the IL, may then be hydrolyzed with a cellulase system 500. The IL may be recovered from the wash-solvent and any dissolved biomass components from the wash-step through suitable separation methods including at least one of the following: activated charcoal treatment, distillation, membrane separation, electrochemical separation techniques, solid phase extraction, liquid-liquid extraction, or a combination thereof. The ionic liquid may then be recycled back to the treatment tank. The wash solvent also may be recycled back for reuse in washing IL-incubated biomass. The wash solvent may also be dehydrated by RF heating to dehydrate the wash solvent, driving off the water leaving a dehydrated IL 900.

The IL may be recovered from the IL/wash solvent mixtures by evaporation of the wash solvent (ethanol and/or water) from the extremely low volatility IL 400. The recovered IL may then be used with no additional cleaning steps in subsequent biomass treatment cycles at constant treatment conditions. The method allows for the repeated reuse of the IL with minimal cleaning which may lead to increased cost savings in IL-treatment.

Residual water in the recycled IL can lower the IL's capacity to sever the inter- and intra-chain hydrogen bonds imparting crystallinity to cellulose. In order to affect swelling of biomass, several of the cellulosic hydrogen-bonds have to be disrupted. Accordingly, it is expect dissolved water to affect IL's performance as a biomass treatment solvent. The admissible water content in IL can affect the economics of the treatment method in two aspects. First, it determines how dry the IL has to be before it can be reused. Second, it determines how dry the biomass has to be during incubation with IL.

After hydrolysis 500, enzymes may be recovered from the hydrolysis reactor and recycled. Complete removal of wash solvent (water) is not necessary before the IL is recycled. Many other treatment methods are not amenable to easy recovery of the chemicals employed in the process. Following the wash of treated biomass 400, lignin from the wash 601 is separated from the hydrolysate residue 600. Also, ultra-filtration of the liquid portion of the hydrolysate, provides a means of recovering the hydrolysis enzymes for reuse from the sugar solution which is the precursor for the production of a number of fuels and chemicals 800.

The current method of treatment with RF and ionic liquid, optionally, followed by hydrolysis (saccharification technique) 500 allows for recovering the lignin in the biomass 600 in the form a post saccharification solid residue. Finally, the lignin obtained following treatment of biomass 300 and/or hydrolysis 500 may be collected and converted by hydrothermal processing 700 to chemicals 701 no further conditioning and adverse effects from any residual traces of IL in the hydrolysate. Further chemical/biochemical processing of the lignin may lead to compounds which could be used for the production of fuels, chemicals, polymers and other materials.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

Although methods and materials similar or equivalent to those described herein may be used in the invention or testing of the present invention, suitable methods and materials are described herein. The materials, methods and examples are illustrative only, and are not intended to be limiting.

The invention now being generally described, it will be more readily understood by reference to the following examples, which are included merely for purposes of illustration of certain aspects and embodiments of the present invention, and are not intended to limit the invention.

### EXAMPLES

### Example 1

### Extraction and Processing of Lignin from Biomass

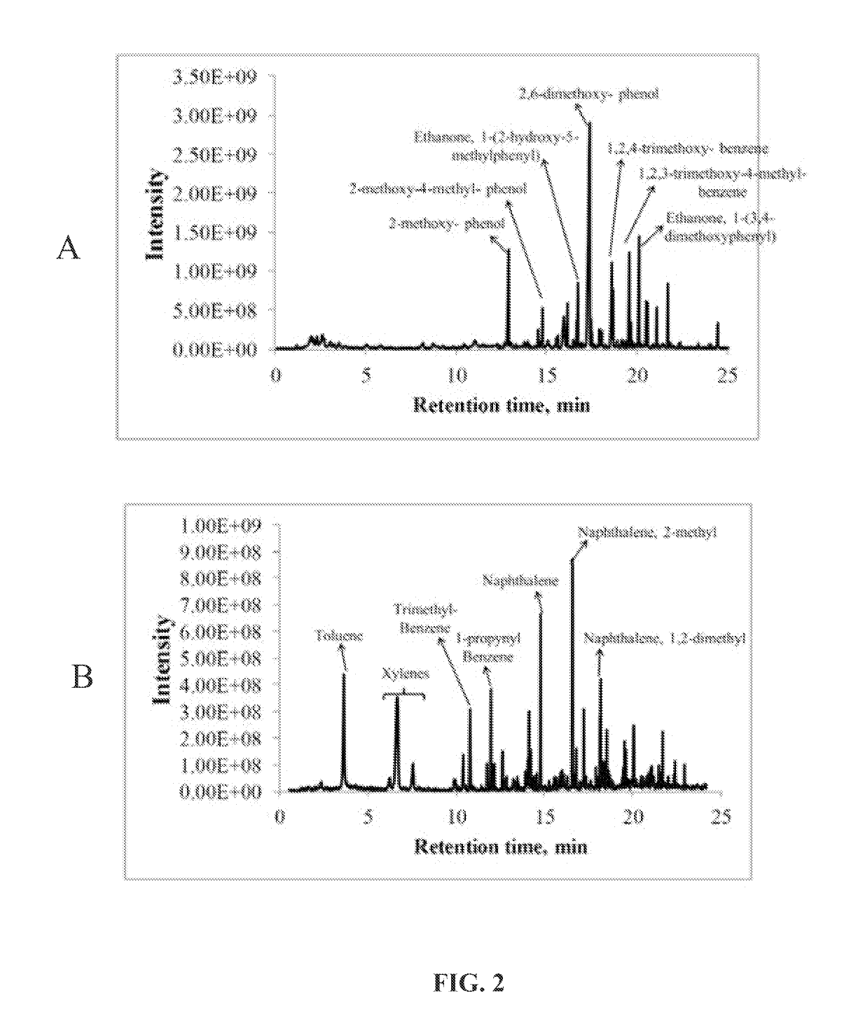

Lignocellulosic biomass was treated by sequential ionic liquid pretreatment followed by hydrolysis to produce a cellulosic fraction and a lignin fraction. See, e.g., U.S. Pat. No. 8,030,030 and U.S. Provisional Patent Application No. 61/663,315. The lignin fraction was subjected to hydrothermal processing about 100-150 ATM and 200-300° C. for approximately 1-30 minutes. The monomeric compounds obtained were analyzed using gas chromatography—mass spectrometry. The results are shown in Tables 1 and 2 and FIGS. 2A and 2B. Products obtained as shown in FIG. 2A are further reprocessed catalytically to produce deoxygenated chemicals as shown in FIG. 2B.

TABLE 1

| S. No | Compound Name |
|---|---|
| 1 | Toluene |
| 2 | Phenol |
| 3 | Phenol, 2-methyl |
| 4 | Phenol, 3-methyl |
| 5 | Indan, 1-methyl |
| 6 | Phenol, 2-methoxy |
| 7 | Phenol, 4-methoxy-3-methyl |
| 8 | Naphthalene |
| 9 | 2-methoxy-5-methylphenol |
| 10 | Phenol,2-methoxy-4-methyl |
| 11 | 3,4-dimethoxytoluene |
| 12 | Phenol, 3,4-dimethoxy |
| 13 | 1,2-Benzenediol, 3-methoxy |
| 14 | Phenol, 4-ethyl-2-methoxy |
| 15 | Naphthalene, 2-methyl |
| 16 | Naphthalene, 1-methyl |
| 17 | 2-methoxy-4-vinylphenol |
| 18 | Benzene, 4-ethyl-1,2-dimethoxy |
| 19 | 1,2,4-trimethoxybenzene |
| 20 | Phenol, 2,6-dimethoxy |
| 21 | 3-allyl-6-methoxyphenol |
| 22 | Phenol, 2-methoxy-4-propyl |
| 23 | Naphthalene, 1-ethyl (or 2-ethyl) |
| 24 | Vanillin |
| 25 | Benzene, 1,2,3-trimethoxy,5-methyl |
| 26 | Phenol,2-methoxy-4-(1-propenyl) |
| 27 | Biphenylene |
| 28 | 3-Hydroxy-4-methoxybenzoic acid |
| 29 | Acenaphthene |
| 30 | Ethanone, 1-(2,6-dihydroxy-4-methoxyphenyl) |
| 31 | 1-Isopropenylnaphthalene |
| 32 | Hexadecane |
| 33 | Phenol, 2,6-dmethoxy-4-(2-propenyl) |
| 34 | Phenol, 2,6-dimethoxy-4-(2-propenyl) |
| 35 | Benzaldehyde, 4-hydroxy-3,5-dimethoxy |
| 36 | 8-Heptadecene |
| 37 | Benzeoic acid, 3,4,5-trimethoxy-, methyl ester |
| 38 | Ethanone, 1-(4-hydroxy-3,5-dimethoxyphenyl) |
| 39 | Anthracene |
| 40 | Phenanthrene, 1-methyl |
| 41 | Anthracene, 1-methyl |
| 42 | Phenanthrene, 1-methyl |
| 43 | Anthracene, 9-ethyl |
| 44 | Phenanthrene, 4,5-dimethyl |
| 45 | Fluoranthene |
| 46 | Pyrene |
| 47 | Acephenanthrylene, 4,5-dihydro |
| 48 | Benzo[k]fluoranthene |
| 49 | Stigmastan-3,4-dien |
| 50 | 9,10-anthracenedione, 1,8-dichloro |
| 51 | Benzo[ghi]perylene |
| 52 | Coronene |
| 53 | 1-hydroxy-2-butanone |
| 54 | 2-Furanmethanol |
| 55 | Butyrolactone |
| 56 | 1H-Imidazole, 1-methyl |
| 57 | Phenol, 2-methoxy |
| 58 | 1,2-Benzenediol, 3-methoxy |
| 59 | 2-methoxy-4-vinylphenol |
| 60 | Phenol, 2,6-dimethoxy |
| 61 | Phenol, 3,4-dimethoxy |
| 62 | 3-hydroxy-4-methoxybenzoic acid |
| 63 | Benzaldehyde, 4-hydroxy-3,5-dimethoxy |
| 64 | Phenol, 2,6-dimethoxy-4-(2-propenyl) |
| 65 | Ethanone, 1-(4-hydroxy-3,5dimethoxyphenyl) |
| 66 | 2-Pentanone, 1-(2,4,6-trihydroxyphenyl) |
| 67 | Butyrolactone |
| 68 | 1H-Imidazole, 1-methyl |
| 69 | Phenol, 2-methoxy |
| 70 | 1,2-Benzenediol, 3-methoxy |
| 71 | Phenol, 4-ethyl-2-methoxy |
| 72 | 2-methoxy-4-vinylphenol |
| 73 | Phenol,2,6-dimethoxy |
| 74 | Phenol, 3,4-dimethoxy |
| 75 | 3-hydroxy-4-methyoxy-benzoic acid |
| 76 | 4-methyl-2,5-dimethoxybenzaldehyde |
| 77 | Phenol, 2,6-dimethyoxy-4-(2-propenyl) |
| 78 | Benzaldehyde, 4-hydroxy-3,5-dimethoxy |
| 79 | Phenol, 2,6-dimethyoxy-4-(2-propenyl) |
| 80 | Ethanone,1-(4-hydroxy-3,5-dimethoxyphenyl) |
| 81 | 2-pentanone, 1-(2,4,6-trihydroxyphenyl) |

TABLE 2

| S. No | Compound |
|---|---|
| 1 | 1-propanol, 2 methoxy |
| 2 | Butyrolactone |
| 3 | Pentanoic Acid 4 oxo methyl ester |
| 4 | Hexanal 2-ethyl |
| 5 | Phenol, 2 methoxy |
| 6 | Phenol 2 methoxy-4 methyl |
| 7 | 1,4-Benzenediol, 2-methoxy |
| 8 | Pnenol 4-ethyl 2 methoxy |
| 9 | Phenol, 2,6-dimethoxy |
| 10 | Phenol, 2-etmoxy-4 propyl |
| 11 | 1,3-benzenediol 4 ethyl |
| 12 | Vanillin |
| 13 | Benzoic Acid, 4-hydroxy-3methoxy |
| 14 | 1,3-Benzenediol, 4 propyl |
| 15 | Ethanone,1-(4-hydroxy-3-methoxy phenyl) |
| 16 | Benzene, 1,2,3-Trimethoxy-5 methyl |
| 17 | 2 Propanone,1-(4-hydroxy-3-methoxy phenol |
| 18 | Homovanillyl Alcohol |
| 19 | 3,4 Dimethoxyphenyl acetone |
| 20 | Benzeneacetic acid, 4-hydroxy 3 methoxy |
| 21 | Vanillacetic acid |
| 22 | Ethyl homovanillate |
| 23 | Ethanone 1-(4-hydroxy-3,5-dimethoxy phynyl) |
| 24 | Phenol, 2-methoxy-4-propyl |

The organic compounds fractionated are surprisingly pure. Thus, they are valuable byproducts of lignin purification and can be commercially utilized to decrease the cost of the overall process and constitute a use for the lignin fraction.

Those skilled in the art will recognize, or be able to ascertain using no more than routine experimentation, many equivalents to the specific embodiments of the invention described herein. Such equivalents are intended to be encompassed by the following claims.

I claim:

1. A method for treating a lignocellulosic biomass comprising incubating a lignocellulosic biomass comprising lignin, cellulose, and hemicellulose in an ionic liquid (IL) for a sufficient time and temperature to swell the cellulose and hemicellulose by without dissolution of the biomass in the IL; washing the IL-incubated biomass comprising lignin, cellulose and hemicellulose with a liquid non-solvent for cellulose that is miscible with water and the IL; and contacting said swelled washed biomass comprising lignin, cellulose and hemicellulose with an aqueous buffer comprising enzymes capable of hydrolyzing both cellulose and hemicellulose to produce polysaccharides; recovering the lignin; and converting said lignin to chemicals.

2. A method for extracting a monomeric compound from a lignin comprising
(a) mixing a biomass with an ionic liquid (IL) to swell said biomass and not dissolve said biomass in IL;
(b) washing said treated biomass;
(c) hydrolysis of said treated biomass;
(d) separating the cellulosic and lignin fractions; and
(e) subjecting the lignin fraction to hydrothermal processing.

3. The method of claim 2, wherein said method further comprises electromagnetic (EM) heating of said swelled biomass after step (a).

4. A method for conversion of the lignin of lignocellulosic biomass to chemicals comprising
(a) mixing biomass in an ionic liquid (IL) to swell said biomass and not dissolve said biomass in IL;
(b) applying radio frequency (RF) heating to the swelled biomass to heat to a target temperature range;
(c) applying ultrasonics, electromagnetic (EM), convective, conductive heating, or combinations thereof, to the swelled biomass to maintain the biomass at said target temperature range;
(d) washing the treated biomass;
(e) separating the cellulosic and lignin fractions; and
(f) subjecting the lignin fraction to hydrothermal processing.

5. The method of claim 4, wherein said target temperature range is about 50-220° C.

6. A method for disruption of the structure of a lignocellulosic biomass comprising incubating a biomass in an ionic liquid (IL) and applying radiofrequency (RF) heating and ultrasonics, electromagnetic (EM), convective, conductive heating, or combinations thereof; washing the treated biomass; recovering the lignin; and subjecting the lignin fraction to hydrothermal processing.

7. A method for conversion of the lignin of lignocellulosic biomass to chemicals comprising hydrothermal processing of lignin.

8. The method of any one of claims 1-7, wherein said lignocellulosic biomass is agricultural residue, wood and forest residue, kudzu, red algae, herbaceous energy crop, plant biomass, or mixtures thereof.

9. The method of claim 8, wherein the agricultural residue is corn stover, wheat straw, bagasse, rice hulls, or rice straw.

10. The method of claim 8, wherein the wood and forest residue is pine, poplar, Douglas fir, oak, saw dust, paper/pulp waste, or wood fiber.

11. The method of claim 8, wherein the herbaceous energy crop is switchgrass, reed canary grass, or miscanthus.

12. The method of any one of claims 1-11, wherein the hydrothermal processing comprises increased pressure.

13. The method of claim 12, wherein said increase pressure is at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 50-100, or 50-150 ATM.

14. The method of claim 12, wherein the pressure may be about 10-100, 20-80, 10-120, or 70-120 ATM.

15. The method of any one of claims 1-11, wherein said hydrothermal processing comprises increased temperature.

16. The method of claim 15, wherein said temperature is about 100, 110, 120, 130, 140, 150, 160, 170, 180, 190, 200, 210, 220, 230, 240, 250, 260, 270, 280, 290, 300, 200-250, 200-300, or 250-300° C.

17. The method of claim 15, wherein the temperature may be about 100-300 ° C., 100-350 ° C., 200-300 ° C., 250-350 ° C., or 300-350 ° C.

18. The method of any one of claims 1-11, wherein said hydrothermal processing is for about 1, 2, 3, 4, 5, 6, 7, 8, 9, 10, 11, 12, 13, 14, 15, 16, 17, 18, 19, 20, 10-20, 10-15, or 1-10 minutes.

19. The method of any one of claims 1-11, wherein said hydrothermal processing is for about 0-3, 1-3, 3-10, 1-10, or 10-15 minutes.

20. The method of any one of claims 1-11, wherein said hydrothermal processing comprises conversion in presence of aromatic or aliphatic alcohols/acids under mild acidic or basic medium.

21. The method of any one of claims 1-11, wherein said hydrothermal processing is catalytically conducted in a single or sequential steps to produce oxygenated products, deoxygenated products and/or dehydrogenated products.

22. The method of claim 1-19, wherein said method further comprises treating said lignin to convert the lignin to its constituent monomers and chemicals.

23. The method of claim 1-19, wherein said method further comprises chemical analysis of said constituent monomers and chemicals.

24. The method of claim 23, wherein said chemical analysis is gas chromatography—mass spectrophotometry, gas chromatography—infrared spectroscopy, liquid chromatography—mass spectrometry, liquid chromatography—NMR spectroscopy, or liquid chromatography -infrared spectroscopy.

25. The method of claim 22, wherein the monomer is toluene; phenol; phenol, 2-methyl; phenol, 3-methyl; indan, 1-methyl; phenol, 2-methoxy; phenol, 4-methoxy-3-methyl; naphalene; 2-methoxy-5-methylphenol; phenol, 2-methoxy-4-methyl; 3,4-dimethoxytoluene; phenol, 3,4-dimethoxy; 1,2-Benzenediol, 3-methoxy; Phenol, 4-ethyl-2-methoxy; Naphthalene, 2-methyl; Naphthalene, 1-methyl; 2-methoxy-4-vinylphenol; Benzene, 4-ethyl-1,2-dimethoxy; 1,2,4-trimethoxybenzene; Phenol, 2,6-dimethoxy; 3-allyl-6-methoxyphenol; Phenol, 2-methoxy-4-propyl; Naphthalene, 1-ethyl (or 2-ethyl); Vanillin; Benzene, 1,2,3-trimethoxy,5-methyl; Pheno1,2-methoxy-4-(1-propenyl); Biphenylene; 3-Hydroxy-4-methoxybenzoic acid; Acenaphthene; Ethanone, 1-(2,6-dihydroxy-4-methoxyphenyl); 1-Isopropenylnaphthalene; Hexadecane; Phenol, 2,6-dmethoxy-4-(2-propenyl); Phenol, 2,6-dimethoxy-4-(2-propenyl); Benzaldehyde, 4-hydroxy-3,5-dimethoxy; 8-Heptadecene; Benzeoic acid, 3,4,5-trimethoxy-, methyl ester; Ethanone, 1-(4-hydroxy-3,5-dimethoxyphenyl); Anthracene; Phenanthrene, 1-methyl; Anthracene, 1-methyl; Phenanthrene, 1-methyl; Anthracene, 9-ethyl; Phenanthrene, 4,5-dimethyl; Fluoranthene; Pyrene; Acephenanthrylene, 4,5-dihydro; Benzo[k]fluoranthene; Stigmastan-3,4-dien; 9,10-anthracenedione, 1,8-dichloro; Benzo[ghi]perylene; Coronene; 1-hydroxy-2-butanone; 2-Furanmethanol; Butyrolactone; 1H-Imidazole, 1-methyl; Phenol, 2-methoxy; 1,2-Benzenediol, 3-methoxy; 2-methoxy-4-vinylphenol; Phenol, 2,6-dimethoxy; Phenol, 3,4-dimethoxy; 3-hydroxy-4-methoxybenzoic acid; Benzaldehyde, 4-hydroxy-3,5-dimethoxy; Phenol, 2,6-dimethoxy-4-(2-propenyl); Ethanone, 1-(4-hydroxy-3,5dimethoxyphenyl); 2-Pentanone, 1-(2,4,6-trihydroxyphenyl); Butyrolactone; 1H-Imidazole, 1-methyl; Phenol, 2-methoxy; 1,2-Benzenediol, 3-methoxy; Phenol, 4-ethyl-2-methoxy; 2-methoxy-4-vinylphenol; Pheno1,2,6-dimethoxy; Phenol, 3,4-dimethoxy; 3-hydroxy-4-methyoxy-benzoic acid; 4-methyl-2,5-dimethoxybenzaldehyde; Phenol, 2,6-dimethyoxy-4-(2-propenyl); Benzaldehyde, 4-hydroxy-3,5-dimethoxy; Phenol, 2,6-dimethyoxy-4-(2-propenyl); Ethanone,1-(4-hydroxy-3,5-dimethoxyphenyl); 2-pentanone, 1-(2,4,6-trihydroxyphenyl); or combinations thereof.

26. The method of claim 22, wherein the monomer is 1-propanol, 2 methoxy; Butyrolactone;
Pentanoic Acid 4 oxo methyl ester; Hexanal 2-ethyl; Phenol, 2 methoxy; Phenol 2 methoxy-4 methyl; 1,4-Benzenediol, 2-methoxy; Phenol 4-ethyl 2 methoxy; Phenol, 2,6-dimethoxy; Phenol, 2-etmoxy-4 propyl; 1,3-benzenediol 4 ethyl; Benzoic Acid, 4-hydroxy-3methoxy; 1,3-Benzenediol, 4 propyl; Ethanone,1-(4-hydroxy-3-methoxy phenyl); Benzene, 1,2,3-Trimethoxy-5 methyl; 2 Propanone,1-(4-hydroxy-3-methoxy phenol; Homovanillyl Alcohol; 3,4 Dimethoxyphenyl acetone; Benzeneacetic acid, 4-hydroxy 3 methoxy; Vanillacetic acid; Ethyl homovanillate; Ethanone 1-(4-hydroxy-3,5-dimethoxy phynyl); Phenol, 2-methoxy-4-propyl; or combinations thereof.

27. The method of claim 22, wherein said chemical is phenol, guaiacol, syringol, eugenol, catechol, vanillin, vanillic acid, syringaldehyde, benzene, toluene, xylene, styrene, biphenyl, cyclohexane, or combinations thereof.

28. The method of any one of claims 1-27, wherein the biomass is subjected to additional heating with agitation, ultrasonics heating, electromagnetic (EM) heating, convective heating, conductive heating, microwave irradiation, or a combination thereof.

29. The method of any one of claims 1-27, wherein said electromagnetic (EM) heating is radiofrequency (RF) heating.

30. The method of any one of claims 1-27, wherein heating comprises at least two phases, a first phase comprising application of electromagnetic (EM) heating, variable frequency heating, radiofrequency (RF) heating, or a combination thereof, and a second phase comprising application of ultrasonics, electromagnetic (EM), convective, conductive heating, or combinations thereof.

31. The method of claim 30, wherein said first phase comprises a variable frequency in the electromagnetic spectrum.

32. The method of any one of claims 1-31, wherein said application of radiofrequency heating is for about at least 5-10 seconds, 1-30 minutes, 5-30 minutes, or 20-240 minutes.

33. The method of claim 32, wherein said application of ultrasonics, electromagnetic (EM), convective, conductive heating, or combinations thereof, is for about at least 3-30 minutes, 5-30 minutes, or 3-4 hours.

34. The method of any one of claims 1-31, wherein said electromagnetic energy is applied at a power of 100-1000W, 1KW-10KW, or 5KW-1MW.

35. The method of any one of claims 1-31, wherein said radiofrequency comprises a frequency between about 1-900 MHz, 300 kHz-3 MHz, 3-30 MHz, 30-300 MHz, 13, 13.56, 27, 27.12, 40, or 40.68 MHz.

36. The method of any one of claims 1-31, wherein said radiofrequency penetrates the biomass to about 0.001 to 2.0 meters thickness.

37. The method of any one of claims 1-31, wherein said biomass is heated to a temperature of at least about 1-300° C., 50° C-100° C., 60° C-130° C., 80° C-175° C., or 100° C-240° C.

38. The method of any one of claims 1-31, wherein said biomass is treated with radiofrequency for at least about 1 minute to 100 hours, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

39. The method of any one of claims 1-38, wherein said method further comprises washing the treated biomass.

40. The method of claim 39, wherein said washing comprises washing the biomass with a liquid non-solvent for cellulose that is miscible with water and the ionic liquid (IL).

41. The method of claim 40, wherein the liquid non-solvent used for washing is water, an alcohol, acetonitrile or a solvent which dissolves the IL and thereby extracts the IL from the biomass.

42. The method of claim 41, wherein the alcohol is ethanol, methanol, butanol, propanol, or mixtures thereof.

43. The method of claim 40, wherein said ionic liquid is recovered from the liquid non-solvent by a method selected from one or more of activated charcoal treatment, distillation, membrane separation, electro-chemical separation techniques, sold-phase extraction liquid-liquid extraction, or a combination thereof.

44. The method of claim 39, wherein said ionic liquid is recovered from the liquid non-solvent by application of electromagnetic heating.

45. The method of claim 44, wherein said ionic liquid is recovered from the liquid non-solvent by application of radiofrequency heating, that dehydrates the ionic liquid.

46. The method of any one of claims 1-45, the method may further comprise reusing the recovered IL for treating more biomass.

47. The method of claim 46, wherein at least 90, 91, 92, 93, 94, 95, 96, 97, 98, or 99% of the IL is recovered.

48. The method of any one of claims 1-47, wherein the ionic liquid has a water content not exceeding about 1, 2, 3, 4, 5, 6, 7, 8, 9, 10, 11, 12, 13, 14, 15, 16, 17, 18, 19, 20, 21, 22, 23, 24, or 25%.

49. The method of any one of claims 1-47, wherein the biomass is subjected to additional heating with intermittent agitation during heating.

50. The method of any one of claims 1-49, wherein the ionic liquid is molten at a temperature ranging from about 10° C. to 160° C. and comprises cations or anions.

51. The method of any one of claims 1-49, wherein the ionic liquid comprises a cation structure that includes ammonium, sulfonium, phosphonium, lithium, imidazolium, pyridinium, picolinium, pyrrolidinium, thiazolium, triazolium, oxazolium, or combinations thereof.

52. The method of claim 51, wherein the ionic liquid comprises a cation selected from imidazolium, pyrrolidinium, pyridinium, phosphonium, ammonium, or a combination thereof.

53. The method of any one of claims 1-49, wherein the ionic liquid (IL) is 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

54. The method of any one of claims 1-53, wherein said method is a continuous process.

55. The method of any one of claims 1-53, wherein said method is a batch process.

56. The method of any one of claims 1-55, wherein the conditions of said biomass undergoing radiofrequency (RF) heating is monitored by sensors.

57. The method of claim 56, wherein, the conditions of said biomass undergoing RF heating is monitored by a liquid flow rate sensor, thermocouple sensor, temperature sensor, salinity sensor, or combinations thereof.

58. The method of any one of claims 1-57, wherein said method comprises adjusting the amount of ionic liquid, the time of incubation, or the temperature of the biomass.

59. The method of any one of claims 1-58, wherein said method further comprises treating said treated lignocellulosic biomass with biochemical reagents.

60. The method of claim 59, wherein said biochemical reagent is an enzyme.

61. The method of claim 60, wherein said enzyme converts the cellulose and hemicellulose to sugar.

62. The method of claim 61, wherein said sugar is a hexose and pentose sugar.
